(19) United States
(12) Patent Application Publication
(10) Pub. No.: US 2008/0138383 A1
(43) Pub. Date: Jun. 12, 2008

(54) COMPOSITIONS AND METHODS FOR TREATING SEIZURES

(76) Inventors: Jonathan David Bortz, Saint Louis, MO (US); R. Saul Levinson, Chesterfield, MO (US); Jeremy Donald Wang, Frontenac, MO (US); Jisheng Ge, Chesterfield, MO (US)

Correspondence Address: KV PHARMACEUTICAL COMPANY, 4080B WEDGEWAY COURT, EARTH CITY, MO, 63045, US

(21) Appl. No.: 11/950,989

(22) Filed: Dec. 5, 2007

Related U.S. Application Data
(60) Provisional application No. 60/869,067, filed on Dec. 7, 2006.

(51) Int. Cl. A61K 31/5513; A61K 31/4164; A61K 9/00; A61P 25/08; A61K 31/505

(52) U.S. Cl. 424/434; 514/221; 514/269; 514/386

(57) ABSTRACT

This invention relates generally to pharmaceutical compositions for treating seizures, and, more particularly, to pharmaceutical compositions that are bioadherent to oral and/or nasal mucosa, comprise one or more anti-acute seizure agents, and can be used to treat one or more conditions selected from the group consisting of acute seizure, repetitive seizures, and status epilepticus. This invention also relates generally to methods for preparing such compositions, methods of treatment using such compositions, uses of such compositions to prepare medicaments, and kits comprising such compositions.

This application claims the benefit of U.S. provisional patent application Ser. No. 60/869,067, filed on Dec. 7, 2006, the entire disclosure of which is incorporated by reference herein.

### FIELD OF THE INVENTION

This invention relates generally to pharmaceutical compositions for treating seizures, and, more particularly, to pharmaceutical compositions that are bioadherent to oral and/or nasal mucosa, comprise one or more anti-acute seizure agents, and can be used to treat one or more conditions selected from the group consisting of acute seizure, repetitive seizures, and status epilepticus. This invention also relates generally to methods for preparing such compositions, methods of treatment using such compositions, uses of such compositions to prepare medicaments, and kits comprising such compositions.

### BACKGROUND OF THE INVENTION

A seizure is defined as an abnormal, disorderly discharging of the brain's nerve cells, resulting in temporary disturbance of motor, sensory, and/or mental function. A seizure can be provoked by, for example, head injury, intoxication with drugs, fever, metabolic disturbances, drug toxicity, infection, withdrawal symptoms, or space-occupying lesions in the brain. Alternatively, a seizure can be unprovoked. Unprovoked seizures are most often associated with epilepsy, a condition in which a person has recurrent seizures.

There are many types of seizures, depending primarily on what part of the brain is involved. An acute seizure typically terminates on its own within about 2 minutes. Repetitive seizures (also known as cluster seizures) are a series of seizures in which the patient regains consciousness between seizures. Repetitive seizures can also be acute repetitive seizures. Status epilepticus is either a prolonged seizure or, alternatively, two or more sequential seizures without full recovery of consciousness between seizures. A prolonged seizure typically lasts at least about 2 minutes, more typically at least about 5 minutes, more typically at least about 10 minutes, more typically at least about 15 minutes, more typically at least about 20 minutes, and even more typically at least about 30 minutes.

One undergoing a seizure can suffer a variety of injuries, for example, bruises, cuts, broken arms, and anoxia-related complications. Therefore, when a seizure takes place, particularly a seizure with extensive tonic-clonic duration (e.g., status epilepticus), it is necessary to provide prompt treatment to moderate or inhibit the seizure to minimize or prevent injury to the patient.

Anti-acute seizure agents can be administered intravenously for acute inhibition of seizures. Intravenous administration may, however, be undesirable in patients undergoing involuntary convulsions because a patient's uncontrolled movements may hinder injection and cause injuries. Further, intravenous administration of diazepam, a preferred anti-acute seizure drug, can be painful and may also cause thrombophlebitis. Intravenous administration is not available to patients and non-medical caregivers outside of a hospital setting. And although intravenously administered drugs typically have a short onset of action, they also typically have a short duration of action, thus necessitating administration of follow-up drugs to achieve complete seizure relief and/or prevent recurrent seizures.

Anti-acute seizure drugs can be administered intramuscularly. Intramuscularly administered anti-seizure drugs typically have a variable onset and duration of action.

Anti-acute seizure drugs can be administered rectally in the form of, for example, suppositories, gels, and liquids. Rectal suppositories are typically slow-acting, and therefore not effective for rendering fast relief of acute seizures. Anti-acute seizure drugs that are rectally administered in the form of gels or liquids typically have an intermediate onset and duration of action. A diazepam-containing rectal gel (Diastat®) has been approved for treating acute seizures. Studies have demonstrated clinical equivalence of rectally and intravenously administered diazepam, but have also shown that the rectal route is not always reliable due to variable bioavailability and wide range of diazepam serum concentrations. There has also been a hesitation to use rectally administered anti-acute seizure drugs because of the need to remove the patient's clothing (often in public places) and the embarrassment that some patients associate with rectal administration.

Anti-acute seizure drugs can be administered in the form of oral tablets. However, it is impractical to administer oral tablets to patients with no voluntary control of skeletal muscle. In addition, anti-acute seizure drugs administered as oral tablets typically have a slow onset of action, thus rendering them less effective in inhibiting acute seizures.

Anti-acute seizure drugs can also be administered to the oral mucosa in the form of a liquid (e.g., in the form of the solution used for intravenous administration), for example, via the sublingual or buccal route of administration. Oral mucosa provides a route into the systemic circulation that is less intrusive and typically easier to access in acute seizures than rectal mucosa. In acute seizures though, it may be impossible to easily or safely lower the patient's jaw to open the oral cavity, and therefore it may be less desirable to use the sublingual route of administration. While it is recommended to maintain the patient's head in neutral position during administration to facilitate absorption, this recommendation is difficult (and often impossible) to follow in convulsing patients. In addition, administering a liquid in the mouth of a convulsing patient can result in choking.

Anti-acute seizure drugs can be administered intranasally. Like administration to the oral mucosa, administration to the nasal mucosa can result in swallowing a lot of liquid, thus increasing the patient's risk for choking. In addition, nose capacity is limited (up to about 0.25 ml per nostril), thus necessitating the use of concentrated drugs and/or repeated applications. Intranasal administration may not be suitable for patients with concurrent upper respiratory tract infection because nasal secretions may dilute the drug solution and interfere with the absorbing surface. Intranasally-administered midazolam has been used for treating acute seizures. The commercially available formulation (Versed®) is acidic and can cause mild nasal irritation; it is not very concentrated and typically results in a lot of liquid being swallowed thereby increasing the patient's risk of choking, making it difficult to determine the optimal dose, or necessitating a second dose.

There is a need for alternative compositions suitable for treating an acute seizure, repetitive seizures, and status epilepticus that are socially acceptable, easy to administer by non-medical caregivers, and capable of minimizing or preventing a patient's risk of choking. This invention provides such compositions as well as methods for treating an acute seizure, repetitive seizures, and status epilepticus utilizing these compositions.

### SUMMARY OF THE INVENTION

This invention relates to pharmaceutical compositions that are bioadherent to human oral and/or nasal mucosa. The compositions comprise one or more anti-acute seizure agents, and are suitable for treating one or more conditions selected from the group consisting of acute seizure, repetitive seizures, and status epilepticus.

This invention also relates to methods for treating one or more conditions selected from the group consisting of acute seizure, repetitive seizures, and status epilepticus. The methods comprise administering a composition described above to the oral and/or nasal mucosa of a patient in need of such treatment.

This invention further relates to uses of the compositions of this invention to prepare medicaments that can be used to treat one or more conditions selected from the group consisting of acute seizure, repetitive seizures, and status epilepticus.

This invention further relates to kits suitable for treating one or more conditions selected from the group consisting of acute seizure, repetitive seizures, and status epilepticus. The kits comprise a composition described above.

Further benefits of applicants' invention will be apparent to one skilled in the art from reading this patent application.

### DETAILED DESCRIPTION OF THE INVENTION

This detailed description is intended only to acquaint others skilled in the art with applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

The compositions of this invention are suitable for treating one or more conditions selected from the group consisting of acute seizure, repetitive seizures, and status epilepticus in a human in need of such treatment. The term “treating” as used in this patent application means ameliorating, suppressing, eradicating, preventing, reducing the risk of, and/or delaying the onset of the condition being treated. In other words, the compositions of this invention are suitable for breaking an acute seizure, a repetitive seizures episode, and/or status epilepticus, and, optionally, also for preventing the occurrence of a subsequent seizure(s). In some embodiments, the compositions can treat the condition(s) being treated while reducing or eliminating the patient's risk of choking. The term “choking” as used in this patent application means obstruction of the flow of air from the environment into the patient's lungs.

In some embodiments, the compositions are suitable for treating an acute seizure.

In some embodiments, the compositions are suitable for treating repetitive seizures. In some such embodiments, the repetitive seizures comprise acute repetitive seizures.

In some embodiments, the compositions are suitable for treating status epilepticus.

Seizures are sudden, transitory, and uncontrolled episodes of brain dysfunction resulting from abnormal discharge of neuronal cells that result in disturbance of motor, sensory, or behavioral changes. Seizures can have a variety of causes, for example, head injury, intoxication with drugs, high fever, metabolic disturbances, drug toxicity, infection, withdrawal symptoms, and space-occupying lesions in the brain. Seizures can also be idiopathic (i.e., occurring without known cause). Such seizures typically occur in people who suffer from epilepsy. It is estimated that about 2.5 million Americans suffer from more than 40 forms of epilepsy. Epileptic seizures can be partial (also known as focal) or generalized seizures. Partial seizures can be simple or complex partial seizures. Generalized seizures can be generalized tonic-clonic, absence, tonic, atonic, and clonic and myoclonic seizures.

Acute seizures typically terminate on their own within about 2 minutes, but can also last a longer period of time. Repetitive seizures are a series of seizures in which the patient regains consciousness between seizures. Such seizures can also be acute repetitive seizures. Status epilepticus is either two or more sequential seizures without full recovery of baseline consciousness between seizures, or alternatively, a prolonged seizure. Status epilepticus can lead to systemic hypoxia, acidemia, hyperpyrexia, cardiovascular arrest, and renal shutdown. The duration of status epilepticus can vary from less than about 5 minutes to at least about 5 minutes, at least about 10 minutes, at least about 15 minutes, at least about 20 minutes, or at least about 30 minutes. A prolonged seizure typically lasts at least about 2 minutes, more typically at least about 5 minutes, more typically at least about 10 minutes, more typically at least about 15 minutes, more typically at least about 20 minutes, and even more typically at least about 30 minutes.

The compositions of this invention are generally targeted release compositions. Specifically, they topically deliver at least a substantial portion of the active ingredient(s) to a specific region, organ, or tissue, and, more particularly, to human oral and/or nasal mucosa. Mucosal drug delivery is an alternative method of systemic drug delivery, and it offers several advantages over injectable and enteral drug delivery methods. Because the mucosa is highly vascularized, drugs absorbed through the mucosa directly enter the systemic circulation, thus bypassing the gastrointestinal tract and first-pass metabolism in the liver. This can lead to rapid onset of action via a more comfortable and convenient delivery route than the intravenous route. The terms “active ingredient” and “drug” as used in this patent application mean an ingredient responsible for a composition's pharmacological activity.

In some embodiments, the compositions of the invention deliver at least a substantial portion of the active ingredient(s) to the oral mucosa.

In some embodiments, the compositions deliver at least a substantial portion of the active ingredient(s) to nasal mucosa.

In some embodiments, an active ingredient (e.g., an anti-acute seizure agent) is released over at least about 1 minute after topical administration of a composition of this invention to the oral and/or nasal mucosa. In other embodiments, an active ingredient is released over at least about 10 minutes after topical administration of the composition to the oral and/or nasal mucosa. In other embodiments, an active ingredient is released over at least about 30 minutes after topical administration of the composition to the oral and/or nasal mucosa. In other embodiments, an active ingredient is released over at least about an hour after topical administration of the composition to the oral and/or nasal mucosa. In other embodiments, an active ingredient is released over at least about 3 hours after topical administration of the composition to the oral and/or nasal mucosa. In yet other embodiments, an active ingredient is released over at least about 6 hours after topical administration of the composition to the oral and/or nasal mucosa. In further embodiments, an active ingredient is released over at least about 12 hours after topical administration of the composition to the oral and/or nasal mucosa. In yet further embodiments, an active ingredient is released over at least about 24 hours after topical administration of the composition to the oral and/or nasal mucosa. Release of the active ingredient(s) will fade at some point after application, which is often no greater than about 2 days after topical administration of the composition to the oral and/or nasal mucosa.

When a composition of this invention comprises two or more active ingredients (e.g., two anti-acute seizure agents), the different active ingredients can have different release periods and/or release profiles. The term “release period” as used in this patent application means a period during which an active ingredient is available for absorption and pharmacological effect (e.g., to treat an acute seizure) after topical administration to the oral and/or nasal mucosa. In other words, the release period of an active ingredient begins when release of the active ingredient substantially begins (e.g., substantially immediately after administration or at a later time if the active ingredient is formulated for delayed release) and ends when substantially no further active ingredient is available for release.

In some embodiments, the compositions of this invention comprise two anti-acute seizure agents, a first anti-acute seizure agent and a second anti-acute seizure agent, wherein the second anti-acute seizure agent has a release period that does not overlap with the release period of the first anti-acute seizure agent. In other embodiments, the compositions comprise two anti-acute seizure agents, a first anti-acute seizure agent and a second anti-acute seizure agent, wherein the second anti-acute seizure agent has a release period that overlaps with the release period of the first anti-acute seizure agent. In some such embodiments, the second anti-acute seizure agent has a release period that overlaps with at least about ¾ of the release period of the first anti-acute seizure agent. In other such embodiments, the second anti-acute seizure agent has a release period that overlaps with at least about ½ of the release period of the first anti-acute seizure agent. In further such embodiments, the second anti-acute seizure agent has a release period that overlaps with at least about ¼ with the release period of the first anti-acute seizure agent. In yet further such embodiments, the second anti-acute seizure agent has a release period that overlaps with at least a terminal portion of the release period of the first anti-acute seizure agent.

In some of the above embodiments, the release profile of the second anti-acute seizure agent is a delayed release profile. In other words, in these embodiments, the release period of the second anti-acute seizure agent begins later (and in some embodiments, substantially later) than the beginning of the release period of the first anti-acute seizure agent. In other of the above embodiments, the release profile of the second anti-acute seizure agent is an extended release profile. In these embodiments, the release period of the second anti-acute seizure agent is longer (and in some embodiments, substantially longer) than the release period of the first anti-acute seizure agent; optionally, the release period of the second anti-acute seizure agent begins later (and in some embodiments, substantially later) than the beginning of the release period of the first anti-acute seizure agent.

In some embodiments, the compositions comprise two anti-acute seizure agents, a first anti-acute seizure agent and a second anti-acute seizure agent, wherein the second anti-acute seizure agent has a release period that begins after release of the first anti-acute seizure agent has peaked. In such embodiments, the second anti-acute seizure agent can exhibit delayed release, and optionally, extended release. In some such embodiments, the second anti-acute seizure agent has a release period which begins after the release period for the first anti-acute seizure agent is substantially complete, so that there is substantially no overlap in the release periods of the first anti-acute seizure agent and the second anti-acute seizure agent.

Similarly to the compositions discussed above, a composition of this invention can comprise a single active ingredient formulated to have two or more different release periods or profiles. In some such embodiments, an active ingredient is formulated in different phases of a composition, with the release periods of the active ingredient formulated in one phase differing from the release period of the active ingredient in another phase.

By utilizing active ingredient(s) with different release periods in a single composition of this invention, the composition can achieve different pharmacological effects such as, for example, moderate an acute seizure as well as prevent the occurrence of future seizure(s). Another benefit of such compositions is that two or more medications can be administered at once.

In some embodiments, the compositions of this invention are bioadherent. The term “bioadherent composition” as used in this patent application means a composition which, after topical administration to human oral and/or nasal mucosa, has a retention time on the mucosal surface that is longer than the retention time of water on that surface. In other words, bioadherent compositions generally adhere to the mucous membranes lining the oral and/or nasal cavity (i.e., to the oral and/or nasal mucosa) and/or display physical stability for a variable period of time after topical administration. Thus, such compositions generally lower or altogether prevent a patient's risk of choking and/or are capable of releasing active ingredient(s) over predetermined periods of time.

In some embodiments, the compositions of this invention generally adhere to the mucous membranes lining the oral cavity (i.e., the compositions are bioadherent to the oral mucosa). In some such embodiments, the compositions generally adhere to the mucous membranes lining the cheek(s) (i.e., the buccal mucosa). In some such embodiments, the compositions generally adhere to the mucous membranes covering the tooth-bearing border of the jaw (i.e., the gingival mucosa). In some such embodiments, the compositions generally adhere to the sublingual mucosa. In some such embodiments, the compositions generally adhere to the mucous membranes lining the roof of the mouth (i.e., the palatal mucosa).

In some embodiments, the compositions generally adhere to the mucous membranes lining the nasal cavity (i.e., the compositions are bioadherent to the nasal mucosa).

In some embodiments, the compositions of this invention adhere to the oral and/or nasal mucosa over at least about 1 minute after topical administration of the composition. In other embodiments, the compositions adhere to the oral and/or nasal mucosa over at least about 5 minutes after topical administration of the composition. In other embodiments, the compositions adhere to the oral and/or nasal mucosa over at least about 10 minutes after topical administration of the composition. In other embodiments, the compositions adhere to the oral and/or nasal mucosa over at least about 20 minutes after topical administration of the composition. In other embodiments, the compositions adhere to the oral and/or nasal mucosa over at least about 30 minutes after topical administration of the composition. In yet other embodiments, the compositions adhere to the oral and/or nasal mucosa over at least about an hour after topical administration of the composition. In other embodiments, the compositions adhere to the oral and/or nasal mucosa over at least about 3 hours after topical administration of the composition. In further embodiments, the compositions adhere to the oral and/or nasal mucosa over at least about 6 hours after topical administration of the composition. In further embodiments, the compositions adhere to the oral and/or nasal mucosa over at least about 12 hours after topical administration of the composition. In yet further embodiments, the compositions adhere to the oral and/or nasal mucosa over at least about 24 hours after topical administration of the composition.

In some embodiments, the compositions of this invention adhere to the oral and/or nasal mucosa from about 1 to at least about 10 minutes after topical administration of the composition to the oral and/or nasal mucosa. In some embodiments, the compositions adhere to the oral and/or nasal mucosa from about 5 to at least about 20 minutes after topical administration. In some embodiments, the compositions adhere to the oral and/or nasal mucosa from about 10 to at least about 30 minutes after topical administration. In some embodiments, the compositions adhere to the oral and/or nasal mucosa from about 20 to at least about an hour after topical administration. In some embodiments, the compositions adhere to the oral and/or nasal mucosa from about 30 minutes to at least about 3 hours after topical administration. In some embodiments, the compositions adhere to the oral and/or nasal mucosa from about 1 to at least about 6 hours after topical administration. In some embodiments, the compositions adhere to the oral and/or nasal mucosa from about 3 to at least about 12 hours after topical administration. In some embodiments, the compositions adhere to the oral and/or nasal mucosa from about 6 to at least about 24 hours after topical administration.

In some embodiments, the compositions of this invention display physical stability over at least about 1 minute after topical administration of the composition to the oral and/or nasal mucosa. In other embodiments, the compositions display physical stability over at least about 10 minutes after topical administration of the composition to the oral and/or nasal mucosa. In other embodiments, the compositions display physical stability over at least about 30 minutes after topical administration of the composition to the oral and/or nasal mucosa. In yet other embodiments, the compositions display physical stability over at least about an hour after topical administration of the composition to the oral and/or nasal mucosa. In yet other embodiments, the compositions display physical stability over at least about 3 hours after topical administration of the composition to the oral and/or nasal mucosa. In yet other embodiments, the compositions display physical stability over at least about 6 hours after topical administration of the composition to the oral and/or nasal mucosa. In further embodiments, the compositions display physical stability over at least about 12 hours after topical administration of the composition to the oral and/or nasal mucosa. In yet further embodiments, the compositions display physical stability over at least about 24 hours after topical administration of the composition to the oral and/or nasal mucosa. The compositions of this invention disintegrate at some point after topical application to the oral and/or nasal mucosa, which point is typically no later than about 2 days after administration.

In some embodiments, the compositions of this invention display physical stability from about 1 to at least about 10 minutes after topical administration of the composition to the oral and/or nasal mucosa. In some embodiments, the compositions display physical stability from about 5 to at least about 20 minutes after topical administration of the composition to the oral and/or nasal mucosa. In some embodiments, the compositions display physical stability from about 10 to at least about 30 minutes after topical administration of the composition to the oral and/or nasal mucosa. In some embodiments, the compositions display physical stability from about 20 to at least about an hour after topical administration of the composition to the oral and/or nasal mucosa. In some embodiments, the compositions display physical stability from about 30 minutes to at least about 3 hours after topical administration of the composition to the oral and/or nasal mucosa. In some embodiments, the compositions display physical stability from about 1 to at least about 6 hours after topical administration of the composition to the oral and/or nasal mucosa. In some embodiments, the compositions display physical stability from about 3 to at least about 12 hours after topical administration of the composition to the oral and/or nasal mucosa. In some embodiments, the compositions display physical stability from about 6 to up to about 24 hours after topical administration of the composition to the oral and/or nasal mucosa.

In some embodiments, a substantial portion of the compositions of this invention disintegrate in the nasopharyngeal cavity after topical administration of the composition to the nasal mucosa. In some such embodiments, a substantial portion of the compositions disintegrate in the nasal cavity after topical administration of the composition to the nasal mucosa.

In other embodiments, a substantial portion of the compositions of this invention disintegrate in the nasopharyngeal cavity after topical administration of the composition to the nasal mucosa. In some such embodiments, a substantial portion of the compositions disintegrate in the nasal cavity after topical administration of the composition to the nasal mucosa.

The compositions of this invention comprise one or more anti-acute seizure agents in addition to the other ingredient(s) of the composition. The term “anti-acute seizure agent” as used in this patent application means an agent suitable, either alone or together with additional medication(s), to treat one or more conditions selected from the group consisting of acute seizure, repetitive seizures, and status epilepticus. An anti-acute seizure agent can comprise a compound or a pharmaceutically acceptable salt of the compound (including solvate or hydrate). Suitable anti-acute seizure agents include the so-called classical anti-seizure agents as well as miscellaneous other anti-seizure agents. The classical anti-seizure agents are classified into four subclasses: barbiturates (e.g., methylphenobarbital, phenobarbital, hexibarbital), hydantoins (e.g., phenyloin, mephenyloin, ethotoin), oxazolidinediones (e.g., trimethdione), and succinimides (e.g., phensuximide, ethoxisuximide). Additional suitable anti-acute seizure agents include, for example, benzodiazepines (e.g., diazepam, lorazepam, midazolam, clonazepam, clorazepate), carbamazepine, primidone, valproic acid, fosphenyloin, felbamate, gabapentin, lamotrigine, tiagabine, zonisamide, topiramate, phenacetamide, and vigabatrin.

The benzodiazepines generally promote the binding of γ-aminobutyric acid (“GABA”) to the GABAA subtype of GABA receptors, which exist as multisubunit, ligand-gated chloride channels, thereby enhancing the GABA-induced ionic currents through these channels. Virtually all effects of the benzodiazepines result from their actions on the central nervous system. The most prominent of these effects are sedation, hypnosis, decreased anxiety, muscle relaxation, anterograde amnesia, and anticonvulsant activity.

The benzodiazepines are well absorbed after oral administration, and their plasma concentrations are usually maximal within 1 to 4 hours. After intravenous administration, they are redistributed in a manner typical of that for highly lipid-soluble agents. Although central effects develop promptly, they also wane rapidly as the drugs move to other tissues. Diazepam is redistributed especially rapidly, with a half-life of redistribution of about 1 hour. The extent of binding of benzodiazepines to plasma proteins correlates with their lipid solubility. For example, approximately 99% of diazepam is bound to plasma proteins. The half-life of diazepam in plasma is between 1 and 2 days. Diazepam has several pharmacologically active metabolites. Its main active metabolite, desmethyldiazepam, is somewhat less active than the parent drug, and has a longer half-life in plasma.

Lorazepam is typically rapidly and nearly completely absorbed after any mode of administration. The onset of action is several minutes after intravenous administration, from about 30 to about 45 minutes after oral/sublingual administration, and up to about 1 hour after intramuscular administration. The duration of action depends on the dose, and is normally from about 6 to about 12 hours. The half-life of lorazepam in patients with normal liver function is typically from about 11 to about 18 hours. There are estimates that approximately 0.5 mg of lorazepam is equivalent to 5 mg of diazepam. Patients treated with lorazepam need to be closely monitored for respiratory depression and hypotensive effects.

Unlike other benzodiazepines such as diazepam and lorazepam, midazolam is water soluble because the imidazoline ring is open at pH under about 4. However, when it is injected, the slightly alkaline (pH about 7.4) environment of the bloodstream causes the imidazoline ring to close, and it becomes much more lipid soluble, facilitating its rapid uptake into nerve tissue. This, and the fact that it has a pKa of about 6.15 and is therefore predominantly unionized at physiological pH, accounts for its rapid onset of action. The elimination half-life of midazolam is usually between about 1.5 and about 3.5 hours.

The hydantoins are chemical compounds which have substituent groups bonded to a hydantoin ring skeletal structure. Phenyloin has two phenyl groups substituted onto the number 5 carbon in a hydantoin molecule. Phenyloin is related to the barbiturates in chemical structure, but has a five-membered ring. Phenyloin typically controls seizures without causing the sedation effects associated with the barbiturate phenobarbital.

Phenobarbital is the oldest anticonvulsant still in use, and is indicated in the treatment of all types of seizures except absence seizures. Phenobarbital is no less effective at seizure control than more modern drugs; it is, however, significantly less well tolerated. Sedation and hypnosis are the principal side effects of phenobarbital; and dizziness, nystagmus, and ataxia are also common. Phenobarbital can cause excitement and confusion in older patients, while it can cause paradoxical hyperreactivity in children. Typically, the first line drugs for treatment of acute seizures and status epilepticus are fast acting benzodiazepines. If these fail, then phenyloin or phenobarbital is typically used.

As discussed above, the compositions of this invention can comprise one or more anti-acute seizure agents. In some embodiments, the anti-acute seizure agents are independently selected from the group consisting of barbiturates, hydantoins, oxazolidinediones, and succinimides. In some embodiments, the anti-acute seizure agents are independently selected from the group consisting of diazepam, lorazepam, midazolam, clonazepam, clorazepate, phenobarbital, methylphenobarbital, hexibarbital, phenyloin, mephenyloin, ethotoin, fosphenyloin, trimethdione, phensuximide, ethoxisuximide, carbamazepine, primidone, valproic acid, felbamate, gabapentin, lamotrigine, tiagabine, zonisamide, topiramate, phenacetamide, vigabatrin, and their pharmaceutically available salts. In some embodiments, the anti-acute seizure agents are independently selected from the group consisting of benzodiazepines, hydantoins, and pharmaceutically available salts thereof. In some embodiments, the anti-acute seizure agents are independently selected from the group consisting of diazepam, lorazepam, midazolam, clonazepam, clorazepate, phenobarbital, methylphenobarbital, hexibarbital, phenyloin, mephenyloin, ethotoin, fosphenyloin, and their pharmaceutically available salts. In some embodiments, the anti-acute seizure agents are independently selected from the group consisting of diazepam, lorazepam, midazolam, phenyloin, phenobarbital, and their pharmaceutically acceptable salts. In some embodiments, the anti-acute seizure agents are independently selected from the group consisting of diazepam, lorazepam, midazolam, clonazepam, and their pharmaceutically acceptable salts. In some embodiments, the anti-acute seizure agents are independently selected from the group consisting of diazepam, lorazepam, midazolam, and their pharmaceutically acceptable salts.

In some embodiments, the compositions of this invention comprise one anti-acute seizure agent. In some such embodiments, the anti-acute seizure agent is selected from the group consisting of diazepam, lorazepam, midazolam, clonazepam, clorazepate, phenyloin, mephenyloin, ethotoin, fosphenyloin, and their pharmaceutically available salts. In other such embodiments, the anti-acute seizure agent is selected from the group consisting of diazepam, lorazepam, midazolam, clonazepam, clorazepate, and their pharmaceutically available salts. In other such embodiments, the anti-acute seizure agent is selected from the group consisting of diazepam, lorazepam, midazolam, and pharmaceutically acceptable salts thereof. In other such embodiments, the agent comprises diazepam or a pharmaceutically acceptable salt thereof. In yet other such embodiments, the anti-acute seizure agent comprises lorazepam or a pharmaceutically acceptable salt thereof. In yet other such embodiments, the anti-acute seizure agent comprises midazolam or a pharmaceutically acceptable salt thereof. In yet other such embodiments, the anti-acute seizure agent comprises clonazepam or a pharmaceutically acceptable salt thereof. In further such embodiments, the anti-acute seizure agent is selected from the group consisting of phenyloin, mephenyloin, ethotoin, fosphenyloin, and their pharmaceutically available salts. In yet further such embodiments, the agent comprises phenyloin or a pharmaceutically acceptable salt thereof.

In some embodiments, the compositions comprise two anti-acute seizure agents. The two anti-acute seizure agents are independently selected. In some such embodiments, the first anti-acute seizure agent is selected from the group consisting of diazepam, lorazepam, midazolam, clonazepam, clorazepate, and pharmaceutically acceptable salts thereof, and the second anti-acute seizure agent is selected from the group consisting of phenobarbital, methylphenobarbital, phenyloin, mephenyloin, ethotoin, fosphenyloin, and pharmaceutically acceptable salts thereof. In other such embodiments, the first anti-acute seizure agent is selected from the group consisting of diazepam, lorazepam, midazolam, clonazepam, clorazepate, and pharmaceutically acceptable salts thereof, and the second anti-acute seizure agent is selected from the group consisting of phenyloin, phenobarbital, and pharmaceutically acceptable salts thereof. In other such embodiments, the first anti-acute seizure agent is selected from the group consisting of diazepam, lorazepam, midazolam, and pharmaceutically acceptable salts thereof, and the second anti-acute seizure agent is selected from the group consisting of phenyloin, phenobarbital, and pharmaceutically acceptable salts thereof. In other such embodiments, the first anti-acute seizure agent comprises diazepam or a pharmaceutically acceptable salt thereof, and the second anti-acute seizure agent comprises phenyloin or a pharmaceutically acceptable salt thereof. In other such embodiments, the first anti-acute seizure agent comprises lorazepam or a pharmaceutically acceptable salt thereof, and the second anti-acute seizure agent comprises phenyloin or a pharmaceutically acceptable salt thereof. In other such embodiments, the first anti-acute seizure agent comprises midazolam or a pharmaceutically acceptable salt thereof, and the second anti-acute seizure agent comprises phenyloin or a pharmaceutically acceptable salt thereof. In other such embodiments, the first anti-acute seizure agent comprises diazepam or a pharmaceutically acceptable salt thereof, and the second anti-acute seizure agent comprises phenobarbital or a pharmaceutically acceptable salt thereof. In further such embodiments, the first anti-acute seizure agent comprises lorazepam or a pharmaceutically acceptable salt thereof, and the second anti-acute seizure agent comprises phenobarbital or a pharmaceutically acceptable salt thereof. And in yet further such embodiments, the first anti-acute seizure agent comprises midazolam or a pharmaceutically acceptable salt thereof, and the second anti-acute seizure agent comprises phenobarbital or a pharmaceutically acceptable salt thereof.

In some embodiments, the compositions comprise one or more anti-acute seizure agents, and at least one of the anti-acute seizure agents has a release period that begins substantially immediately after topical administration to the oral and/or nasal mucosa. In some such embodiments, that anti-acute seizure agent is selected from the group consisting of diazepam, lorazepam, midazolam, clonazepam, clorazepate, and pharmaceutically acceptable salts thereof. In other such embodiments, that anti-acute seizure agent is selected from the group consisting of diazepam, lorazepam, midazolam, and pharmaceutically acceptable salts thereof.

In some embodiments, the compositions comprise two anti-acute seizure agents (i.e., a first anti-acute seizure agent and a second anti-acute seizure agent), and the second anti-acute seizure agent has a release period whose beginning is later (and in some embodiments, substantially later) than the beginning of the release period of first anti-acute seizure agent. In some such embodiments, the first anti-acute seizure agent is selected from the group consisting of diazepam, lorazepam, midazolam, and pharmaceutically acceptable salts thereof. In other such embodiments, the second anti-acute seizure agent is selected from the group consisting of phenyloin, phenobarbital, diazepam, lorazepam, midazolam, and pharmaceutically acceptable salts thereof. In yet other such embodiments, the first anti-acute seizure agent is selected from the group consisting of diazepam, lorazepam, midazolam, and pharmaceutically acceptable salts thereof, and the second anti-acute seizure agent is selected from the group consisting of phenyloin, phenobarbital, diazepam, lorazepam, midazolam, and pharmaceutically acceptable salts thereof.

In some embodiments, the compositions comprise two anti-acute seizure agents (i.e., a first anti-acute agent and a second anti-acute agent), and the second anti-acute seizure agent has a release period that is longer (and in some embodiments) substantially longer than the release period of the first anti-acute seizure agent. In some such embodiments, the first anti-acute seizure agent is selected from the group consisting of diazepam, lorazepam, midazolam, and pharmaceutically acceptable salts thereof. In other such embodiments, the second anti-acute seizure agent is selected from the group consisting of phenyloin, phenobarbital, diazepam, lorazepam, midazolam, and pharmaceutically acceptable salts thereof. In yet other such embodiments, the first anti-acute seizure agent is selected from the group consisting of diazepam, lorazepam, midazolam, and pharmaceutically acceptable salts thereof, and the second anti-acute seizure agent is selected from the group consisting of phenyloin, phenobarbital, diazepam, lorazepam, midazolam, and pharmaceutically acceptable salts thereof.

In some embodiments, the compositions comprise two anti-acute seizure agents (i.e., a first anti-acute seizure agent and a second anti-acute seizure agent), and the second anti-acute seizure agent has a release period that begins later (and in some embodiments, substantially later) than the beginning of the release period of the first anti-acute seizure agent, and is longer (and in some embodiments, substantially longer) than the release period of the first anti-acute seizure agent. In some such embodiments, the first anti-acute seizure agent is selected from the group consisting of diazepam, lorazepam, midazolam, and pharmaceutically acceptable salts thereof. In other such embodiments, the second anti-acute seizure agent is selected from the group consisting of phenyloin, phenobarbital, diazepam, lorazepam, midazolam, and pharmaceutically acceptable salts thereof. In yet other such embodiments, the first anti-acute seizure agent is selected from the group consisting of diazepam, lorazepam, midazolam, and pharmaceutically acceptable salts thereof, and the second anti-acute seizure agent is selected from the group consisting of phenyloin, phenobarbital, diazepam, lorazepam, midazolam, and pharmaceutically acceptable salts thereof.

In some embodiments, the compositions of this invention comprise a hydrophobic external phase and an aqueous internal phase, wherein the aqueous internal phase is encased or dispersed within the hydrophobic external phase. At least one of the phases comprises an active ingredient (i.e., at least one active ingredient is present in at least one phase). In other words, such compositions can comprise an active ingredient in the external phase, internal phase, or both phases. The presence of an active ingredient in a phase depends on, for example, the hydrophobicity or hydrophilicity of the active ingredient, the desired pharmacological profile of the active ingredient, and the type of ingredients in the composition. The desirability of a particular ingredient in a phase depends on, for example, the function of the ingredient, the condition being treated, and the environment in which the composition is being applied (e.g., pH). For example, the compositions of this invention can comprise the same active ingredient in more than one phase. In such embodiments, the active ingredient is released from the different phases at different times and/or over different periods of time (i.e., the active ingredient in one phase has a different release period and/or release profile from the active ingredient in another phase). For example, a substantial portion of the active ingredient present in one phase can be released promptly after topical administration of the composition to the oral and/or nasal mucosa while a substantial portion of the active ingredient present in another phase can be released over an extended period of time following application. The compositions of this invention can also comprise two or more active ingredients that are released from the same or different phases at varying times and/or over varying periods of time. For example, a substantial portion of some active ingredients can be released promptly after topical administration of the composition while a substantial portion of other active ingredients can be released over varying extended periods of time following application.

In some embodiments, the compositions of this invention further comprise one or more therapeutic agents other than anti-acute seizure agents. The therapeutic agents can be used to (a) treat the condition(s) causing the patient's seizure(s); (b) treat any seizure-unrelated conditions that the patient has; and/or (c) to mitigate side-effect(s) of the administered anti-acute seizure agents. Suitable therapeutic agents include, for example, anti-inflammatory agents, anti-hypertensive agents, anti-hypotensive agents, anti-pyretic agents, anti-neoplasia agents, anti-psychotic agents, stimulants, anti-mycotic agents, anti-microbial agents, antibiotics, immunomodulating agents, anti-viral agents (e.g., acyclovir), bronchodilators (e.g., albuterol), anti-thyroid agents, anti-hypoglycemic agents, anti-opioid agents (e.g., naloxone), hormones, hormone antagonists, drugs affecting renal and/or cardiovascular function (e.g., diuretics, vasopressin, renin, anti-arrhythmic drugs), drugs acting on blood forming organs and/or blood clotting mechanism(s), drugs acting on bone calcification and/or turnover, drugs affecting gastrointestinal function, drugs acting on the central and autonomic nervous system (e.g., anticholinergics, adrenergics, anesthetics, parasympathomimetics, sympathomimetics, hypnotics, sedatives, serotonin-like drugs, serotonin blockers), and agents that mitigate the side-effects of the administered anti-acute seizure agents. Suitable anti-inflammatory agents include, for example, NSAIDs (e.g., aspirin, ibuprofen, naproxen, oxaprozin, celecoxib, refecoxib, valdecoxib), histamine antagonists, glucocorticoids (e.g., betamethazone, cortisone, dexamethasone, hydrocortisone, prednisone, prednisolone, triamcinolone), anti-inflammatory cytokines, and anti-histamines (e.g., diphenhydramine, chlorpheniramine, hydroxyzine, azelastine, levocabastine, ketotifen, cetirizine, levocetirizine, loratidine, desloratidine, acrivastine, ebastine, fexofenadine, mizolastine, cycloheptadine, azelastine, betahistine, perceptin, ciproxifan, thioperamide, burimamide, cimetidine, ranitidine, famotidine). Suitable anti-hypertensive agents include, for example, α₁-adrenergic antagonists (e.g., prazocin), β-adrenergic antagonists (e.g., propranolol, nadolol, timolol, metoprolol, pindolol, albuterol), angiotensin converting enzyme inhibitors (e.g., captopril, enalapril, lisinopril), calcium channel blockers (e.g., verapamil, diltiazem, nifedipine), and diuretics (e.g., hydrochlorothiazide, chlorthalidone, furosemide, triamterene). Suitable stimulants include, for example, caffeine and amphetamines (e.g., dextroamphetamine). Suitable anti-pyretic agents include, for example, aspirin and acetaminophen. Suitable anti-hypoglycemic agents include, for example, glucose and sucrose.

In some embodiments, these therapeutic agents cause little or no irritation after topical administration to the oral and/or nasal mucosa.

In some embodiments, the therapeutic agents have low toxicity after topical administration to the oral and/or nasal mucosa.

In some embodiments, the therapeutic agents are effective after topical administration to the oral and/or nasal mucosa.

In some embodiments, the compositions of this invention are prepared as liquid dosage forms, including in situ gelling liquid dosage forms (e.g., solution, emulsion, suspension, syrup, elixir, aerosol, spray, foam, gel). In other embodiments, the compositions are prepared as semisolid dosage forms (e.g., ointment, cream, paste, gel). In further embodiments, the compositions are prepared as solid dosage forms (e.g., film, wafer).

In some embodiments, the compositions comprise a dosage form selected from the group consisting of solution, emulsion, suspension, syrup, elixir, ointment, cream, gel, paste, spray, aerosol, film, and wafer.

In some embodiments, the compositions of this invention comprise a liquid dosage form comprising an emulsion. An “emulsion” is generally a two-phase system in which one liquid is dispersed throughout another liquid in the form of small droplets. In some embodiments, the compositions comprise a liquid dosage form comprising a suspension. A “suspension” generally is a liquid preparation that consists of solid particles dispersed throughout a liquid phase in which the particles are not soluble. In some embodiments, the compositions comprise a liquid dosage form comprising a lotion. In other embodiments, the compositions comprise a liquid dosage form comprising a foam. A “foam” generally is an emulsion packaged in a pressurized aerosol container that has a fluffy, semisolid consistency when released after actuating the aerosol valve.

In some embodiments, the compositions of this invention comprise a solid dosage form. In some such embodiments, the solid dosage form comprises a film. In other such embodiments, the solid dosage form comprises a wafer.

In some embodiments, the compositions of this invention comprise a semisolid dosage form comprising a cream. A “cream” generally is a semisolid dosage form containing one or more substances dissolved or dispersed in a suitable base. In other embodiments, the compositions comprise a semisolid dosage form comprising a gel. A “gel” generally is a liquid or semisolid system consisting of either a suspension of small inorganic particles or large organic molecules interpenetrated by a liquid. In other embodiments, the compositions comprise a semisolid dosage form comprising an ointment. An “ointment” generally is a semisolid preparation intended for external application to the skin or mucous membrane. In other embodiments, the compositions comprise a semisolid dosage form comprising a paste. A “paste” generally is a semisolid dosage form that contains one or more drug substances intended for topical application.

As discussed above, in some embodiments, the compositions of this invention comprise in situ gelling liquid dosage forms. Such in situ gelling compositions comprise one or more gelling agents, and are typically liquid at room temperature. Once applied to the oral and/or nasal mucosa, the viscosity of the compositions increases (due to the exposure to body temperature), and the compositions form gels. The formed gels typically are thermoreversible gels.

Suitable gelling agents include polycarboxylic acid based polymers, for example, poly(acrylic, maleic, itaconic, citraconic, methacrylic) acids and derivatives thereof. Such polymers illustratively include methacrylic acid-ethylacrylate copolymers sold under the Eudragit® brand (e.g., Eudragit® L 100-55). Suitable gelling agents also include cellulose derivatives, for example, methyl-, ethyl-, methylethyl-, hydroxymethyl-, hydroxyethyl-, hydroxypropyl-, carboxymethyl-, and hydroxypropylmethyl-celluloses as well as esters and salts thereof. Suitable gelling agents further include glycosaminoglycans (e.g., hyaluronic acid and derivatives thereof). Suitable gelling agents further include cross-linked acrylic-based polymers modified with alkyl acrylates (also known as carbomer resins and sold under the Carbopol® brand (e.g., Carbopol® 940). Suitable gelling agents further include polyoxyethylene/polyoxypropylene copolymers sold under Lutrol® and Pluronic® brands.

In some embodiments, the compositions comprise a block copolymer consisting of polyoxyethylene and polyoxypropylene units or a mixture of two or more such block copolymers. In some embodiments, a block copolymer has a molecular weight of from about 6840 to about 17400. In some such embodiments, a block copolymer has a molecular weight of from about 6840 to about 8830. In other such embodiments, a block copolymer has a molecular weight of from about 7680 to about 9150. In other such embodiments, a block copolymer has a molecular weight of from about 9840 to about 14600. In yet other such embodiments, a block copolymer has a molecular weight of from about 12700 to about 17400. Suitable block copolymers include, for example, Poloxamer 407 (also known as Lutrol® F127NF or Pluronic®F127NF and sold by BASF), Poloxamer 188, Poloxamer 237, and Poloxamer 338.

In some embodiments, the compositions comprise one or more block copolymers corresponding in structure to the formula

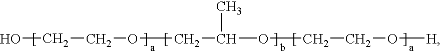

wherein a is an integer from about 25 to about 225, and b is an integer from about 10 to 200. In some such embodiments, a is from about 95 to about 105, and b is from about 50 to about 60. In some of these embodiments, the block copolymer is Poloxamer 407 (a being about 101 and b being about 56). In other such embodiments, a is from about 135 to about 145, and b is from about 40 to about 50. In some of these embodiments, the block copolymer is Poloxamer 338 (a being about 141 and b being about 44). In yet other such embodiments, a is from about 60 to about 70, and b is from about 30 to about 40. In some of these embodiments, the block copolymer is Poloxamer 237 (a being about 64 and b being about 37). In further such embodiments, a is from about 75 to about 85, and b is from about 20 to about 30. In some of these embodiments, the block copolymer is Poloxamer 188 (a being about 80 and b being about 27).

In some of the above embodiments, the compositions comprise from about 3 to about 40% by weight block copolymer(s) (i.e., from about 3 to about 40 g block copolymer(s) (total) per about 100 g of composition). Unless otherwise indicated, all percentages in this patent application are weight percentages. In some such embodiments, the compositions comprise from about 5 to about 15% by weight block copolymer(s). In other such embodiments, the compositions comprise from about 10 to about 25% by weight block copolymer(s). In other such embodiments, the compositions comprise from about 20 to about 25% by weight block copolymer(s). In other such embodiments, the compositions comprise from about 20 to about 40% by weight block copolymer(s).

In some embodiments, the compositions further comprise ethanol. In some such embodiments, the compositions comprise from about 2 to about 60% by weight ethanol. In some such embodiments, the compositions comprise from about 5 to about 15% by weight ethanol, from about 10 to about 20% by weight ethanol, from about 20 to about 25% by weight ethanol, from about 20 to about 30% by weight ethanol, from about 30 to about 40% by weight ethanol, from about 40 to about 50% by weight ethanol, or from about 50 to about 60% by weight ethanol.

In some embodiments, the compositions further comprise sodium chloride. In some such embodiments, the compositions comprise from about 0.01 to about 3% by weight sodium chloride. In some such embodiments, the compositions comprise from about 0.01 to about 0.5% by weight sodium chloride, from about 0.5 to about 1% by weight sodium chloride, from about 1 to about 1.5% by weight sodium chloride, from about 1.5 to about 2% by weight sodium chloride, from about 2 to about 2.5% by weight sodium chloride, or from about 2.5 to about 3% by weight sodium chloride.

In some embodiments, the compositions comprise from about 20 to about 22% by weight Poloxamer 407, from about 20 to about 25% by weight ethanol, and from about 1 to about 2% by weight sodium chloride.

This invention also relates to pharmaceutical compositions formulated in in situ gelling liquid dosage forms that are suitable for administration to human oral and/or nasal mucosa, and comprise one or more active ingredients and one or more gelling agents. In some embodiments, the compositions are bioadherent to human oral and/or nasal mucosa.

Anti-acute seizure agents and combinations of such agents suitable for the gelling compositions of the invention are discussed above. Various active ingredients other than anti-acute seizure agents, which are suitable for the gelling compositions of this invention also are discussed above. Suitable therapeutic agents include, for example, anti-inflammatory agents, anti-hypertensive agents, anti-hypotensive agents, anti-pyretic agents, anti-neoplasia agents, anti-psychotic agents, stimulants, anti-mycotic agents, anti-microbial agents, antibiotics, immunomodulating agents, anti-viral agents, bronchodilators, anti-thyroid agents, anti-hypoglycemic agents, anti-opioid agents, hormones, hormone antagonists, drugs affecting renal and/or cardiovascular function, drugs acting on blood forming organs and/or blood clotting mechanism(s), drugs acting on bone calcification and/or turnover, drugs affecting gastrointestinal function, and drugs acting on the central and autonomic nervous system. The gelling compositions of this invention can comprise any active ingredient or a suitable combination of active ingredients that can be formulated into the compositions.

Various gelling agents suitable for the gelling compositions of this invention are also discussed above. Suitable gelling agents include, for example, polycarboxylic acid based polymers (e.g., Eudragit® brand polymers), cellulose derivatives (e.g., methyl-, ethyl-, methylethyl-, hydroxymethyl-, hydroxyethyl-, hydroxypropyl-, carboxymethyl-, and hydroxypropylmethyl-celluloses as well as esters and salts thereof), glycosaminoglycans (e.g., hyaluronic acid and derivatives thereof), acrylic-based polymers (e.g., Carbopol® brand polymers), and polyoxyethylene/polyoxypropylene polymers (e.g., Lutrol® and Pluronic® brand polymers).

The gelling compositions comprise from about 1 to about 40% by weight gelling agent(s). In some such embodiments, the compositions comprise from about 1 to about 5% by weight gelling agent(s). In other such embodiments, the compositions comprise from about 5 to about 10% by weight gelling agent(s). In other such embodiments, the compositions comprise from about 5 to about 15% gelling agent(s). In other such embodiments, the compositions comprise from about 10 to about 25% gelling agent(s). In other such embodiments, the compositions comprise from about 20 to about 25% gelling agent(s). In other such embodiments, the compositions comprise from about 20 to about 40% gelling agent(s).

In some of the above embodiments, the gelling compositions further comprise ethanol. In some such embodiments, the compositions comprise from about 2 to about 60% by weight ethanol. In some such embodiments, the compositions comprise from about 5 to about 15% by weight ethanol, from about 10 to about 20% by weight ethanol, from about 20 to about 25% by weight ethanol, from about 20 to about 30% by weight ethanol, from about 30 to about 40% by weight ethanol, from about 40 to about 50% by weight ethanol, or from about 50 to about 60% by weight ethanol.

In some of the above embodiments, the gelling compositions further comprise sodium chloride. In some such embodiments, the compositions comprise from about 0.01 to about 3% by weight sodium chloride. In some such embodiments, the compositions comprise from about 0.01 to about 0.5% by weight sodium chloride, from about 0.5 to about 1% by weight sodium chloride, from about 1 to about 1.5% by weight sodium chloride, from about 1.5 to about 2% by weight sodium chloride, from about 2 to about 2.5% by weight sodium chloride, or from about 2.5 to about 3% by weight sodium chloride.

This invention also relates to pharmaceutical compositions that are suitable for administration to human oral and/or nasal mucosa, and comprise one or more active ingredients, from about 20 to about 60% by weight ethanol, and from about 10 to about 25% by weight sorbitol. In some embodiments, the compositions are bioadherent to human oral and/or nasal mucosa.

Anti-acute seizure agents and combinations of such agents suitable for the compositions are discussed above. Various active ingredients other than anti-acute seizure agents, which are suitable for the compositions also are discussed above. For example, such active ingredients include anti-inflammatory agents, anti-hypertensive agents, anti-hypotensive agents, anti-pyretic agents, anti-neoplasia agents, anti-psychotic agents, stimulants, anti-mycotic agents, anti-microbial agents, antibiotics, immunomodulating agents, anti-viral agents, bronchodilators, anti-thyroid agents, anti-hypoglycemic agents, anti-opioid agents, hormones, hormone antagonists, drugs affecting renal and/or cardiovascular function, drugs acting on blood forming organs and/or blood clotting mechanism(s), drugs acting on bone calcification and/or turnover, drugs affecting gastrointestinal function, and drugs acting on the central and autonomic nervous system. The compositions can comprise any active ingredient or a suitable combination of active ingredients that can be formulated into the compositions.

In some embodiments, the compositions comprise from about 25 to about 55% by weight ethanol. In other embodiments, the compositions comprise from about 25 to about 35% by weight ethanol. In further embodiments, the compositions comprise from about 45 to about 55% by weight ethanol.

In some embodiments, the compositions comprise from about 15 to about 20% by weight sorbitol.

In some embodiments, the compositions comprise from about 25 to about 55% by weight ethanol and from about 15 to about 20% by weight sorbitol.

In some of the above embodiments, the compositions further comprise from about 0.01 to about 3% by weight sodium chloride. In some such embodiments, the compositions comprise from about 0.5 to about 1% by weight sodium chloride.

In some embodiments, the compositions of this invention have a pH of from about 2 to about 9. In some such embodiments, the compositions have a pH of from about 3.0 to about 7.5. In other such embodiments, the compositions have a pH of about 6 to about 7.

The compositions of this invention preferably have sufficient viscosity to be bioadherent. In some embodiments, the compositions have a viscosity of up to about 1,200,000 centipoise. In some such embodiments, the compositions have a viscosity of from about 20,000 to about 1,200,000 centipoise. In other such embodiments, the compositions have a viscosity of from about 600,000 to about 1,200,000 centipoise.

In some embodiments, the osmolarity of the water phase of the compositions of this invention is from about 100 to about 600 milliosmoles/liter. In some such embodiments, the osmolarity of the water phase is from about 300 to about 400 milliosmoles/liter. In other such embodiments, the osmolarity of the water phase is from about 290 to about 310 milliosmoles/liter.

The absorption profile of the active ingredient(s) and/or the physical stability of the compositions of this invention can be varied by varying the ratio of the amount of active ingredient(s) in a particular composition to the volume of the composition. The compositions of this invention typically comprise from about 0.1 to about 100 mg active ingredient(s) in a volume of from about 0.1 to about 25 ml. In some embodiments, the compositions comprise from about 5 to about 40 mg active ingredient(s) in a volume of from about 0.1 to about 25 ml. In other embodiments, the compositions comprise from about 5 to about 40 mg active ingredient(s) in a volume of from about 0.1 to about 1.5 ml. In other embodiments, the compositions comprise from about 5 to about 40 mg active ingredient(s) in a volume of from about 1 to about 3 ml. In other embodiments, the compositions comprise from about 5 to about 40 mg active ingredient(s) in a volume of from about 2.5 to about 5 ml. In other such embodiments, the compositions comprise from about 5 to about 40 mg active ingredient(s) in a volume of from about 3 to about 10 ml. In other embodiments, the compositions comprise from about 5 to about 40 mg active ingredient(s) in a volume of from about 7.5 to about 15 ml. Because different active ingredients have different physicochemical and pharmacokinetic properties (e.g., solubility in different solvents, bioavailability, potency), the ratios of the amount of active ingredient(s) to volume can vary greatly from the ratios described in this paragraph. For example, in the case of very potent drugs such as hormones and cytokines, it will be desirable to use much less drug (e.g., from about 0.001 to about 1 mg) for comparable volume of the composition.

The compositions of this invention may be applied to the oral and/or nasal mucosa by any means such as, for example, an applicator, or by spraying and aerosolization.

In some embodiments, a unit dose (i.e., an amount of the composition suitable for a single administration) of the compositions of this invention is provided in a disposable, pre-filled applicator.

In some embodiments, the compositions of this invention are provided in bulk in a suitable container, with a patient or caregiver dispensing the needed dose. In some such embodiments, the compositions are provided with an applicator that can be used for measuring the needed dose and/or applying the composition.

The compositions of this invention typically are applied promptly after symptoms of the treated condition are detected by the patient or a caregiver. The dosage regimen actually employed can vary widely. Factors affecting the preferred dosage regimen include the type, age, weight, sex, and condition of the patient; the severity of the condition; pharmacological considerations, such as the activity, efficacy, pharmacokinetic, and the toxicology profiles of the particular active ingredient(s) used.

The compositions of this invention typically comprise pharmaceutically acceptable carriers, adjuvants, and/or vehicles (together referred to as “excipients”) such as, for example, permeation enhancers, flavoring agents, sweetening agents, suspending agents, preservatives, colorants, anti-foaming agents, buffers, decolorizing agents, and solubilizers.

Suitable permeation enhancers include, for example, fatty acids, fatty acid esters, fatty alcohols, fatty acid esters of lactic acid or glycolic acid, glycerol triesters, glycerol diesters, glycerol monoesters, triacetin, short chain alcohols, and dimethyl sulfoxide. Additional permeation enhancers are listed in, for example, Osborne at al., Pharmaceutical Technology 21:50-66 (1997). Methods for assaying the characteristics of permeation enhancers are known in the art. See, for example, Merritt et al., Journal of Controlled Release 1:161-162 (1984).

Suitable flavoring agents include, for example, spearmint oil, peppermint oil, cinnamon oil, citrus oils, and fruit essences.

Suitable sweetening agents include, for example, artificial sweeteners, and water-soluble mono-, di-, and polysaccharides.

The active ingredient(s) in the compositions of this invention can be used in the form of salts derived from inorganic or organic acids. Depending on the particular drug, a salt of the drug may be advantageous due to one or more of the salt's physical properties, such as enhanced pharmaceutical stability in differing temperatures and humidities, or a desirable solubility in water or oil. Salts for administration to patients preferably are pharmaceutically acceptable.

Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound. Pharmaceutically acceptable acid addition salts of the drugs used in the compositions of this invention may often be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Pharmaceutically acceptable base addition salts of the drugs used in the compositions of this invention include, for example, metallic salts and organic salts.

This invention also relates to methods for treating one or more conditions that can be treated with the compositions of this invention.

In some embodiments, the invention relates to methods for treating a condition selected from the group consisting of acute seizure, repetitive seizures, and status epilepticus in a human in need thereof. As discussed above, the seizure(s) can be idiopathic or have one or more causes, and be epileptic as well as non-epileptic. The methods comprise administering to the oral and/or nasal mucosa of the human an effective amount of a composition of this invention. The composition can be administered before a seizure has started, during a seizure, in between seizures, and/or after a seizure has terminated. The term “effective amount” or “therapeutically-effective amount” as used in this patent application means an amount that will achieve the goal of treating the targeted condition.

In some embodiments, the treated condition comprises acute seizure.

In some embodiments, the treated condition comprises repetitive seizures. In some such embodiments, the treated condition comprises acute repetitive seizures.

In some embodiments, the treated condition comprises status epilepticus.

In some embodiments, the methods of this invention comprise a combination therapy wherein a composition of this invention is co-administered with a second (or even a third, fourth, etc.) composition comprising an active ingredient, such as, for example, anti-inflammatory agent, anti-hypertensive agent, anti-hypotensive agent, fever-reducing agent, stimulant, or agent that mitigates side-effect(s) of the administered anti-acute seizure agent(s). In these embodiments, the composition of this invention and the second composition may be administered in a substantially simultaneous manner (e.g., within about 5 minutes of each other), in a sequential manner, or both. It is contemplated that such combination therapies may include administering one composition multiple times between the administration of the other composition. The time period between the administration of each composition may range from a few seconds (or less) to several minutes or hours, and will depend on, for example, the properties of each composition and active ingredient (e.g., potency, solubility, bioavailability, half-life, and kinetic profile), as well as the condition of the patient.

The second composition may be administered using a dosage form suitable for the active ingredient(s) present in that composition to have an intended effect. Preferred methods of administration for the second composition include ones that are suitable for the condition of the patient undergoing treatment. In general, the second composition comprises from about 0.05 to about 95% by weight of one or more active ingredients, and may be prepared by a variety of well-known techniques of pharmacy that include the step of bringing into association the active ingredient(s) with one or more excipients.

This invention also relates to uses of the compositions of this invention to prepare medicaments. In some embodiments, the medicaments are suitable for treating one or more conditions selected from the group consisting of acute seizure, repetitive seizures, and status epilepticus.

This invention also relates to kits comprising a composition of the invention. In some embodiments, the kits are suitable for treating one or more conditions selected from the group consisting of acute seizure, repetitive seizures, and status epilepticus. These kits comprise a composition of the invention, and optionally instructions for use and/or an applicator. The term “applicator” as used in this patent application encompasses any means for applying the composition to the oral and/or nasal mucosa. In some embodiments, the applicator can also be used to measure the needed dose. In some embodiments, the kit further comprises a second (or even a third, fourth, etc.) composition comprising one or more active ingredients such as, for example, anti-inflammatory agents, anti-hypotensive agents, fever-reducing agents, stimulants, and/or agents that mitigate the side-effects of the administered anti-acute seizure agent(s).

In some embodiments, a composition of this invention is provided in a kit in bulk in a suitable container. In some such embodiments, the composition is provided with applicator(s) which can optionally be used to measure the needed dose. The applicator(s) can be disposable or suitable for multiple uses. In other embodiments, a composition of this invention is provided in the kit in a disposable, pre-filled applicator.

### EXAMPLES

The following examples are merely illustrative, and not limiting to this disclosure in any way.

### Example 1

### Preparation of Formulations 1 and 2

| | Formulation 1 | Formulation 2 |
|---|---|---|
| Propylene Glycol | 10.0% | 3.0% |
| Pluronic ® F127 | 22.0% | 22.0% |
| Edetate Disodium | 0.05% | 0.05% |
| Methylparaben, NF | 0.08% | 0.08% |
| Propylparaben, NF | 0.02% | 0.02% |
| Water | to 100% | to 100% |

The protocol for preparing formulations 1 and 2 is as follows:

1. Weigh water in a glass beaker, add edetate sodium, and mix well to dissolve the edetate sodium.

2. Weigh propylene glycol into another beaker, add methylparaben and propylparaben, and mix well to dissolve the parabens.

3. Combine the solutions from steps 1 and 2.

4. While stirring the solution from step 3, slowly add Pluronic® F127 powder.

### Example 2

### Preparation of Formulations 3 and 4

| | Formulation 3 | Formulation 4 |
|---|---|---|
| Pluronic ® F127 | 14.7% | 14.7% |
| Eudragit ® L 100-55 | 2.0% | 4.0% |
| Water | to 100% | to 100% |

The protocol for preparing formulations 3 and 4 is as follows:

1. Prepare 15% stock solution of Pluronic® F127 by dissolving 63 g material into 357 g of water.

2. Add stock solution into a container first.

3. Slowly add Eudragit® L 100-55 into the solution to dissolve it.

### Example 3

### Preparation of Formulations 5 and 6

| | Formulation 5 | Formulation 6 |
|---|---|---|
| Pluronic ® F127 | 13.4% | 14.0% |
| Carbopol ® 940 | 2.0% | 2.0% |
| Eudragit ® L 100-55 | 4.0% | — |
| Water | to 100% | to 100% |

The protocol for preparing formulations 5 and 6 is as follows:

1. Prepare 14.3% stock solution of Pluronic® F127 by dissolving 50 g material into 300 g of water.

2. Add stock solution into a container first.

3. Then add the polymer(s) (Carbopol® 940 and Eudragit® L 100-55) into above solution slowly to disperse them.

### Example 4

### Preparation of Formulation 7

Formulation 7 comprises diazepam, 0.96%; water, 63%; ethanol, 20%; and a lower concentration of a Poloxamer® (e.g., Lutrol® F127), 16%. The formulation is a free-flowing, liquid sol which will turn into a gel state when warmed to body temperature (e.g., after administration to oral and/or nasal mucosa).

### Example 5

### Preparation of Formulation 8

Formulation 8 comprises diazepam, 0.96%; water, 48%; sodium chloride, 0.9%; ethanol, 34%; and a lower concentration of a Poloxamer® (e.g., Lutrol® F127), 16%. The formulation is a free-flowing, liquid sol which will turn into a gel state when warmed to body temperature (e.g., after administration to oral and/or nasal mucosa).

### Example 6

### Preparation of Formulation 9

Formulation 9 comprises diazepam, 0.96%; water, 51%; sodium chloride, 0.75%; ethanol, 20%; and a higher concentration of a Poloxamer® (e.g., Lutrol® F127), 27%. The formulation is a free-flowing, liquid sol which will turn into a gel state when warmed to body temperature (e.g., after administration to oral and/or nasal mucosa).

### Example 7

### Preparation of Formulation 10

Formulation 10 comprising diazepam, 0.96%; water, 53%; sodium chloride, 1%; ethanol, 23%; and a higher concentration of a Poloxamer® (e.g., Lutrol® F127), 22%, is prepared by first dissolving the Poloxamer in ethanol with a minimum amount of water and later adding the rest of the ingredients. The formulation is a free-flowing, liquid sol which will turn into a gel state when warmed to body temperature (e.g., after administration to oral and/or nasal mucosa).

### Example 8

### Preparation of Formulation 11

Formulation 11 comprising diazepam, 0.96%; water, 53%; sodium chloride, 1%; grain alcohol, 23%; and a higher concentration of a Poloxamer®, 22%, is prepared by first dissolving the Poloxamer in ethanol with a minimum amount of water and later adding the rest of the ingredients. The formulation is a free-flowing, liquid sol which will turn into a gel state when warmed to body temperature (e.g., after administration to oral and/or nasal mucosa).

### Example 9

### Preparation of Formulation 12

Formulation 12 comprising water, 54%; sodium chloride, 1.7%; ethyl alcohol, 22.5%; and a Poloxamer® (e.g., Lutrol® F127), 21.8% is prepared by first dissolving the Poloxamer in ethyl alcohol with a minimum amount of water and later adding the rest of the ingredients. The formulation is a free-flowing, liquid sol (density=1.04 g/mL; viscosity >1,200 cps) which will turn into a gel state when warmed to body temperature (e.g., after administration to oral and/or nasal mucosa).

### Example 10

### Preparation of Formulation 13

Formulation 13 comprising diazepam, 0.96%; water, 53%; sodium chloride, 1.4%; sugar, 2%; ethyl alcohol, 22.5%; a Poloxamer (e.g., Lutrol® F127), 20%; and one or more flavors as needed (e.g., peppermint) is prepared by first dissolving the Poloxamer in ethyl alcohol with a minimum amount of water and later adding the rest of the ingredients. The formulation is a free-flowing, liquid sol which will turn into a gel state when warmed to body temperature (e.g., after administration to oral and/or nasal mucosa).

### Example 11

### Preparation of Formulation 14

Formulation 14 comprising diazepam, 0.96%; water, 53-54%; sodium chloride, ˜1%; sugar, 2%; ethyl alcohol, 20-22%; a Poloxamer® (e.g., Lutrol® F127), 21-22%; one or more flavors as needed (e.g., peppermint); and one or more preservatives as needed (e.g., methylparaben, propylparaben) is prepared by first dissolving the Poloxamer and the flavor and preservatives in ethyl alcohol with a minimum amount of water and later adding the rest of the ingredients. The formulation is a free-flowing, liquid sol which will turn into a gel state when warmed to body temperature (e.g., after administration to oral and/or nasal mucosa).

### Example 12

### Preparation of Formulation 15

Formulation 15 comprising diazepam, 0.96%; water, 32%; sodium chloride, 0.9%; sugar, 2%; ethyl alcohol, 23%; sorbitol, 19%; a Poloxamer® (e.g., Lutrol® F127), 22%; one or more flavors as needed (e.g., peppermint); and one or more preservatives as needed is prepared by first dissolving the active, Poloxamer, the flavor, and preservatives in the ethyl alcohol with a minimum amount of water and later adding the rest of the ingredients. The formulation is a free-flowing, liquid sol which will turn into a gel state when warmed to body temperature (e.g., after administration to oral and/or nasal mucosa).

### Example 13

### Preparation of Formulation 16

Formulation 16 comprising acyclovir, 5%; water, 29%; sodium chloride, 0.9%; sugar, 2%; ethyl alcohol, 22%; sorbitol, 19%; a Poloxamer®, 21%; one or more flavors as needed (e.g., peppermint); and one or more preservatives as needed is prepared by first dissolving the active, Poloxamer, the flavor, and preservatives in the ethyl alcohol with a minimum amount of water and later adding the rest of the ingredients. The formulation is a free-flowing, liquid sol which will turn into a gel state when warmed to body temperature (e.g., after administration to oral and/or nasal mucosa).

### Example 14

### Preparation of Formulation 17

Formulation 17 comprising albuterol sulfate, an active, 0.05%; water, 32%; sodium chloride, 0.9%; sugar, 2%; ethyl alcohol, 23%; a Poloxamer®, 23%; sorbitol, 19%; one or more flavors as needed (e.g., peppermint); and one or more preservatives as needed is prepared by first dissolving the active, Poloxamer, the flavor, and preservatives in the ethyl alcohol with a minimum amount of water and later adding the rest of the ingredients. The formulation is a free-flowing, liquid sol which will turn into a gel state when warmed to body temperature (e.g., after administration to oral and/or nasal mucosa).

### Example 15

### Preparation of Formulation 18

Formulation 18 comprising the active or actives; water, 39-45%; sodium chloride, ˜1%; sugar, 2%; ethyl alcohol, 22-25%; sorbitol, 17-22%; methyl cellulose (or a derivative), 5-10%; one or more flavors as needed; and one or more preservatives as needed is prepared by first dissolving the actives, methyl cellulose, the flavor, and preservatives in the ethyl alcohol with a minimum amount of water and later adding the rest of the ingredients.

### Example 16

### Preparation of Formulation 19

Formulation 19 comprising the active or actives; water, 40-50%; sodium chloride, ˜1%; sugar, 2%; ethyl alcohol, 22-25%; sorbitol, 17-22%; an hyaluronic acid or derivative thereof, 1-5%; one or more flavors as needed; and one or more preservatives as needed is prepared by first dissolving the active, HA, the flavor, and preservatives in the ethyl alcohol with a minimum amount of water and later adding the rest of the ingredients.

### Example 17

### Preparation of Formulation 20

Formulation 20 comprising diazepam, 0.96%; water, 27%; sodium chloride, 0.9%; sugar, 2%; ethyl alcohol, 50%; sorbitol, 19%; one or more flavors as needed; and one or more preservatives as needed is prepared by first dissolving the actives, the flavor, and the preservatives in the ethyl alcohol with a minimum amount of water and later adding the rest of the ingredients.

### Example 18

### Preparation of Formulation 21

Formulation 21 comprising phenobarbital, 1.25%; water, 27%; sodium chloride, 0.9%; sugar, 2%; ethyl alcohol, 49%; sorbitol, 19%; one or more flavors as needed; and one or more preservatives as needed is prepared by first dissolving the actives, the flavor, and the preservatives in the ethyl alcohol with a minimum amount of water and later adding the rest of the ingredients.

### Example 19

### Preparation of Formulation 22

Formulation 22 comprising phenyloin, an active, 20%; water, 29%; sodium chloride, 0.9%; sugar, 2%; ethyl alcohol, 29%; sorbitol, 19%; one or more flavors as needed; and one or more preservatives as needed is prepared by first dissolving the actives, the flavor, and the preservatives in the ethyl alcohol with a minimum amount of water and later adding the rest of the ingredients.

### Example 20

### Preparation of Formulation 23

Formulation 23 comprising phenobarbital, 1.2%; diazepam, 0.96%; water, 29%; sodium chloride, 0.9%; sugar, 2%; ethyl alcohol, 29%; sorbitol, 19%; one or more flavors as needed; and one or more preservatives as needed is prepared by first dissolving the actives, the flavors, and the preservatives in the ethyl alcohol with a minimum amount of water and later adding the rest of the ingredients.

The words “comprise”, “comprises”, and “comprising” are to be interpreted inclusively rather than exclusively.

All references cited above are incorporated by reference into this patent application in their entirety. The discussion of the references is intended merely to summarize assertions made by their authors. No admission is made that any reference (or a portion thereof) is relevant prior art. Applicants reserve the right to challenge the accuracy and pertinence of the cited references.

We claim:

1. A pharmaceutical composition for treating one or more conditions selected from the group consisting of acute seizure, repetitive seizures, and status epilepticus in a human in need of such treatment, wherein the composition comprises one or more anti-acute seizure agents and is bioadherent to human oral and/or nasal mucosa.

2. A pharmaceutical composition for treating one or more conditions selected from the group consisting of acute seizure, repetitive seizures, and status epilepticus in a human in need of such treatment, wherein the composition comprises one or more anti-acute seizure agents, and, after administration to human oral and/or nasal mucosa, can treat the condition(s) while reducing or eliminating the human's risk of choking.

3. The composition of claim 1, wherein the condition is acute seizure.

4. The composition of claim 1, wherein at least a substantial portion of the composition disintegrates in the oropharyngeal cavity after administration.

5. The composition of claim 1, wherein at least a substantial portion of the composition disintegrates in the nasopharyngeal cavity after administration.

6. The composition of claim 1, wherein the one or more anti-acute seizure agents are independently selected from the group consisting of diazepam, lorazepam, midazolam, clonazepam, clorazepate, phenobarbital, methylphenobarbital, hexibarbital, phenyloin, mephenyloin, ethotoin, fosphenyloin, trimethdione, phensuximide, ethoxisuximide, carbamazepine, primidone, valproic acid, felbamate, gabapentin, lamotrigine, tiagabine, zonisamide, topiramate, phenacetamide, vigabatrin, and their pharmaceutically available salts.

7. The composition of claim 1, wherein the composition comprises one anti-acute seizure agent selected from the group consisting of diazepam, lorazepam, midazolam, and pharmaceutically acceptable salts thereof.

8. The composition of claim 1, wherein the composition comprises one anti-acute seizure agent, and the anti-acute seizure agent comprises diazepam or a pharmaceutically acceptable salt thereof.

9. The composition of claim 1, wherein the composition comprises two anti-acute seizure agents, a first anti-acute seizure agent and a second anti-acute seizure agent, the first anti-acute seizure agent is selected from the group consisting of diazepam, lorazepam, midazolam, clonazepam, clorazepate, and pharmaceutically acceptable salts thereof, and the second anti-acute seizure agent is selected from the group consisting of phenobarbital, methylphenobarbital, phenyloin, mephenyloin, ethotoin, fosphenyloin, and pharmaceutically acceptable salts thereof.

10. The composition of claim 1, wherein the composition comprises two anti-acute seizure agents, a first anti-acute seizure agent and a second anti-acute seizure agent, the first anti-acute seizure agent comprises diazepam or a pharmaceutically acceptable salt thereof, and the second anti-acute seizure agent comprises phenyloin or a pharmaceutically acceptable salt thereof.

11. The composition of claim 1, wherein at least one of the one or more anti-acute seizure agents has a release period that begins substantially immediately after administration.

12. The composition of claim 1, wherein the composition comprises two anti-acute seizure agents, a first anti-acute seizure agent and a second anti-acute seizure agent, and the second anti-acute seizure agent has a release period that begins later than the beginning of the release period of the first anti-acute seizure agent.

13. The composition of claim 12, wherein the release period of the first anti-acute seizure agent does not overlap with the release period of the first anti-acute seizure agent.

14. The composition of claim 1, wherein the composition comprises two anti-acute seizure agents, a first anti-acute seizure agent and a second anti-acute seizure agent, and the second anti-acute seizure agent has a release period that is longer than the release period of the first anti-acute seizure agent.

15. The composition of claim 14, wherein the second anti-acute seizure agent has a release period that begins later than the beginning of the release period of the first anti-acute seizure agent.

16. The composition of claim 15, wherein the release period of the first anti-acute seizure agent does not overlap with the release period of the first anti-acute seizure agent.

17. The composition of claim 1, wherein the composition further comprises one or more therapeutic agents other than anti-acute seizure agents.

18. The composition of claim 1, wherein the composition further comprises one or more permeation enhancers.

19. The composition of claim 1, wherein the composition has a pH of from about 3.0 to about 7.5.

20. The composition of claim 1, wherein the composition is in a liquid dosage form.

21. The composition of claim 1, wherein the composition is in a semisolid dosage form.

22. The composition of claim 1, wherein the composition is in a solid dosage form.

23. The composition of claim 20, wherein the composition is in an in situ gelling liquid dosage form.

24. The composition of claim 23, wherein the composition comprises a block copolymer corresponding in structure to the formula 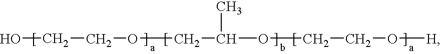
wherein a is an integer from about 25 to about 225 and b is an integer from about 10 to about 200.

25. The composition of claim 23, wherein the composition comprises a block copolymer having molecular weight from about 9840 to about 14600 and consisting of polyoxyethylene and polyoxypropylene units.

26. The composition of claim 23, wherein the composition comprises Poloxamer 407.

27. The composition of claim 23, wherein the composition comprises from about 3 to about 40% by weight Poloxamer 407.

28. The composition of claim 23, wherein the composition comprises from about 5 to about 10% by weight methylcellulose or a derivative thereof.

29. The composition of claim 23, wherein the composition comprises from about 1 to about 5% by weight hyaluronic acid or a derivative thereof.

30. The composition of claim 24, wherein the composition further comprises from about 2 to about 60% by weight ethanol.

31. The composition of claim 24, wherein the composition further comprises from about 0.01 to about 3% by weight sodium chloride.

32. The composition of claim 24, wherein the composition further comprises from about 20 to about 25% by weight ethanol, from about 1 to about 2% by weight sodium chloride, and from about 50 to about 60% by weight water.

33. The composition of claim 23, wherein the composition has osmolarity of from about 290 to about 310 milliosmoles.

34. The composition of claim 1, wherein the composition is in the form of a film or wafer, wherein at least a substantial portion of the film or wafer disintegrates in the oropharyngeal and/or nasopharyngeal cavity after administration.

35. A kit for treating a condition selected from the group consisting of acute seizure, repetitive seizures, and status epilepticus in a human in need of such treatment, wherein the kit comprises a composition of claim 1 and instructions how to administer the composition.

36. The kit of claim 35, wherein the kit further comprises one or more applicators for administering the composition.

37. A method for treating a condition selected from the group consisting of acute seizure, repetitive seizures, and status epilepticus in a human in need of such treatment, wherein the method comprises administering to the oral and/or nasal mucosa of the human an effective amount of a composition of claim 1.

38. A pharmaceutical composition formulated in an in situ liquid gelling form suitable for administration to human oral and/or nasal mucosa, wherein the composition comprises:
one or more active ingredients; and
one or more gelling agents.

39. The composition of claim 38, wherein the composition is bioadherent to human oral and/or nasal mucosa.

40. The composition of claim 38, wherein the one or more active ingredients are independently selected from the group consisting of anti-inflammatory agents, anti-hypertensive agents, anti-hypotensive agents, anti-pyretic agents, anti-neoplasia agents, anti-psychotic agents, stimulants, anti-mycotic agents, anti-microbial agents, antibiotics, immunomodulating agents, anti-viral agents, bronchodilators, anti-thyroid agents, anti-hypoglycemic agents, anti-opioid agents, hormones, hormone antagonists, drugs affecting renal and/or cardiovascular function, drugs acting on blood forming organs and/or blood clotting mechanism(s), drugs acting on bone calcification and/or turnover, drugs affecting gastrointestinal function, drugs acting on the central and autonomic nervous system, and anti-seizure agents.

41. The composition of claim 38, wherein the composition comprises from about 1 to about 40% by weight gelling agent(s).

42. The composition of claim 38, wherein the composition further comprises from about 2 to about 60% by weight ethanol.

43. The composition of claim 38, wherein the composition further comprises from about 0.01 to about 3% by weight sodium chloride.

44. A pharmaceutical composition suitable for administration to human oral and/or nasal mucosa, wherein the composition comprises:
one or more active ingredients;
from about 20 to about 60% by weight ethanol; and
from about 10 to about 25% by weight sorbitol.

45. The composition of claim 44, wherein the composition is bioadherent to human oral and/or nasal mucosa.

46. The composition of claim 44, wherein the one or more active ingredients are independently selected from the group consisting of anti-inflammatory agents, anti-hypertensive agents, anti-hypotensive agents, anti-pyretic agents, anti-neoplasia agents, anti-psychotic agents, stimulants, anti-mycotic agents, anti-microbial agents, antibiotics, immunomodulating agents, anti-viral agents, bronchodilators, anti-thyroid agents, anti-hypoglycemic agents, anti-opioid agents, hormones, hormone antagonists, drugs affecting renal and/or cardiovascular function, drugs acting on blood forming organs and/or blood clotting mechanism(s), drugs acting on bone calcification and/or turnover, drugs affecting gastrointestinal function, drugs acting on the central and autonomic nervous system, and anti-seizure agents.

47. The composition of claim 44, wherein the composition comprises from about 25 to about 55% by weight ethanol.

48. The composition of claim 44, wherein the composition comprises from about 25 to about 35% by weight ethanol.

49. The composition of claim 44, wherein the composition comprises from about 45 to about 55% by weight ethanol.

50. The composition of claim 44, wherein the composition comprises from about 15 to about 20% by weight sorbitol.

51. The composition of claim 44, wherein the composition comprises:
from about 25 to about 55% by weight ethanol; and
from about 15 to about 20% by weight sorbitol.

52. The composition of claim 44, wherein the composition further comprises from about 0.01 to about 3% by weight sodium chloride.

53. The composition of claim 44, wherein the composition comprises from about 0.5 to about 1% by weight sodium chloride.
